(19) United States
(12) Patent Application Publication
(10) Pub. No.: US 2003/0152624 A1
(43) Pub. Date: Aug. 14, 2003

(54) Controlled release dosage form having improved drug release properties

(76) Inventors: Dale S. Aldrich, Richland, MI (US); Randy J. Wald, Portage, MI (US); Anthony J. Hlinak, Lindenhurst, IL (US); Rebanta Bandyopadhyay, Portage, MI (US); Martin J. Coffey, Portage, MI (US); Diane Goll, Portage, MI (US); Ahmad M. Mazhary, Algonquin, IL (US); Richard H. Meury, Kalamazoo, MI (US); Brian R. Rohrs, Scotts, MI (US); John W. Skoug, Portage, MI (US); Stephen L. Secreast, Portage, MI (US); Dennis J. Stelzer, Otsego, MI (US); Jackie G. White, Portage, MI (US)

Correspondence Address: PHARMACIA CORPORATION, GLOBAL PATENT DEPARTMENT, POST OFFICE BOX 1027, ST. LOUIS, MO, 63006, US

(21) Appl. No.: 10/325,298

(22) Filed: Dec. 19, 2002

Related U.S. Application Data
(60) Provisional application No. 60/342,650, filed on Dec. 20, 2001.

(51) Int. Cl. A61K 31/137; A61K 9/22

(52) U.S. Cl. 424/468; 514/649

(57) ABSTRACT

Controlled release pharmaceutical compositions comprising tolterodine and a polymer-based release-controlling component and processes for preparing such compositions are provided. The compositions are useful in the treatment of overactive bladder and similar conditions.

This application claims priority of U.S. provisional application serial No. 60/342,650 filed Dec. 20, 2001.

### FIELD OF THE INVENTION

The present invention relates to improved processes for preparing controlled release dosage forms comprising tolterodine and a polymer-based release-controlling component, and to compositions prepared according to such processes.

### BACKGROUND OF THE INVENTION

Controlled release dosage forms have become an important delivery vehicle for a wide variety of drugs. Such dosage forms provide many potential advantages over traditional dosage forms including, inter alia, increased patient compliance, improved delivery efficiency, decreased total drug requirement, minimization or elimination of local or systemic side effects, and minimization of drug accumulation with chronic dosing.

Typical controlled release dosage forms include coated beads or pellets, coated tablets and ion exchange resins wherein release of the active drug is brought about through selective breakdown of, or permeation through, a coating on the formulation, or through a special matrix which affects drug release.

An important aspect of all controlled release dosage forms relates to the need for consistent drug release between dose units prepared in the same and/or in different production batches, and throughout the shelf life of the finished product. Such release stability requirements are provided for in the Good Manufacturing Practices (GMPs), the United States Pharmacopoeia (USP), in New Drug Applications (NDAs) and Investigational New Drug Applications (INDs).

Hydrophobic polymers such as certain cellulose derivatives, zein, acrulic acrylic resins, waxes, higher aliphatic alcohols, and polylactic and polyglyolic acids have been used in controlled release dosage forms, generally as overcoating materials. Such hydrophobic coating can be applied from solution, suspension, or in dry form.

U.S. Pat. No. 6,129,933 to Oshlack teaches that subjecting a controlled release dosage form which comprises a hydrophobic polymer to a rigorous curing process can reduce changes to the hydrophobic polymer that occur as a result of prolonged storage at elevated temperature and/or humidity levels. This curing step is said to stabilize release of the therapeutic agent upon aging of the finished dosage form.

International Patent Publication No. WO 01/19901 discloses sustained release beadlets of chlorpheniramine maleate, phenylpropanolamine, pseudoephedrine and dextromethorphan; the beadlets are coated with a pseudolatex water swellable polymer dispersion.

Co-assigned International Patent Publication No. WO 00/27364 describes a controlled release bead comprising a core unit of a substantially water-soluble or water-swellable inert material, a first layer on the core unit of a substantially water-insoluble polymer, a second layer covering the first layer and containing an active ingredient; and a third layer of polymer on the second layer effective for controlled release of the active ingredient, wherein the first layer is adapted to control water penetration into the core. A process for preparing such controlled release beads is also disclosed therein. The process provides an optional curing step to prevent drug release properties from changing after production of the beads, during storage and transportation.

Detrol® LA and Detrusitol® SR, both of Pharmacia Corporation, are exemplary once daily dosage forms prepared according to processes described immediately above. Detrol® LA and Detrusitol® SR are clinically important treatments for overactive bladder, a condition which afflicts an estimated 50 million people worldwide.

We have now unexpectedly discovered that compositions described in International Patent Publication No. WO 00/27364 can exhibit undesirable drug release variability. Several adverse consequences result from such variability including, inter alia, decreased production efficiency due to an increased percentage of batches failing to meet acceptable drug release criteria, expensive and time consuming requirement for in-process drug release monitoring, and potential inter batch variability in therapeutic effect of final dosage units. Therefore, there is a significant and heretofore unmet need for controlled release dosage units in which inter-batch drug release rate variability is reduced and for improved processes for preparing such dosage units.

### SUMMARY OF THE INVENTION

There is now provided a controlled release pharmaceutical composition in the form of discrete dosage units having been prepared in a multi-batch process of manufacture. The dosage units comprise tolterodine or a tolterodine-related compound as an active drug and a pharmaceutically acceptable polymer-based release-controlling component. The polymer-based release-controlling component has an age distribution at time of manufacture of the dosage units such that upon randomly sampling a plurality of the dosage units and individually placing each of the sampled dosage units in identical standard in vitro dissolution tests, the sampled dosage units exhibit drug release, measured immediately after 3 hours in the dissolution tests, which varies by not more than about ±15% of a target.

Also provided are processes for preparing such compositions. Compositions of the invention overcome the above-described inter-batch dissolution variability problems in a surprisingly effective and heretofore unexpected manner. Other features of this invention will be in part apparent and in part pointed out hereinafter.

### BRIEF DESCRIPTION OF THE DRAWINGS

FIG. 1 is a flow diagram illustrating a representative method for preparation of a controlled release composition of the invention.

FIG. 2 is a graph showing average in vitro drug release (% of target), at three hours, from test articles prepared in Batches 1-101 of Examples 1-3.

### DETAILED DESCRIPTION OF THE INVENTION

Controlled Release Compositions

The term “controlled release compositions” herein refers to any composition or dosage form which comprises an active drug and which is formulated to provide a longer duration of pharmacological response after administration of the dosage form than is ordinarily experienced after administration of a corresponding immediate release composition comprising the same drug in the same amount. Controlled release compositions include, inter alia, those compositions described elsewhere as “extended release”, “delayed release”, “sustained release”, “prolonged release”, “programmed release”, “time release” and/or “rate controlled” compositions or dosage forms.

Compositions of the invention are preferably in the form of finished dosage units. Preferably, one to a small plurality of such dosage units are suitable to provide a therapeutically and/or prophylactically effective daily dose of active drug. More preferably, 1 or 2 dosage units provide a therapeutically and/or prophylactically effective daily dose of active drug. Illustrative dosage units include tablets, spheroids, beads, microspheres, seeds, pellets, ion-exchange resins, etc. Granules, spheroids, beads, pellets or other like dosage units, if more than one is required to provide a sufficient dose, are preferably presented in a capsule or other container so as to provide a discrete dosage unit. In a particularly preferred embodiment, dosage units of the invention are in the form of controlled release beads encapsulated in a capsule, for example a hard gelatin capsule.

Illustratively, a controlled release dosage unit prepared by a process of the invention provides an in vitro drug release profile, when measured by the USP Paddle Method at 100 rpm in 900 ml aqueous buffer (pH between about 1.6 and about 7.2) at 37° C. characterized as follows: not more than about 30% by weight of labeled amount of therapeutically active agent is released after 1 hour; about 40% to about 85% by weight of labeled amount of therapeutically active agent is released after 3 hours; and not less than about 80% by weight of labeled amount of therapeutically active agent is released after 7 hours. This example is for illustrative purposes only and is not intended to be limiting in any respect.

Active Drug

Compositions and processes of the invention are suitable for any active agent, drug, or compound. The terms “agent”, “drug”, and “compound” herein refer to a therapeutically active ingredient. The drug can be soluble or insoluble in water.

An exemplary class of compounds which may be used as an active agent in processes and compositions of the invention comprises the 3,3-diphenylpropylamines disclosed in the patents cited below, each of which are hereby individually incorporated herein by reference in their entirety.

U.S. Pat. No. 5,382,600.

U.S. Pat. No. 5,559,269.

U.S. Pat. No. 5,686,464.

Such compounds have the general formula:

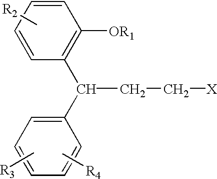

wherein R₁ signifies hydrogen or methyl; R₂, R₃ and R₄ independently signify hydrogen, methyl, methoxyl, hydroxy, hydroxymethyl, carbamoyl, sulfamoyl or halogen; and X represents a tertiary amino group —NR₅R₆, wherein R₅ and R₆ signify non-aromatic hydrocarbyl groups, which may be the same or different, especially C₁₋₆ alkyl or adamantyl, and which together contain at least three, preferably at least four carbon atoms, and each of which may carry a hydroxy substituent, and wherein R₅ and R₆ may form a ring together with the amine nitrogen, preferably a non-aromatic ring having no heteroatom other than the amine nitrogen, their salts with physiologically acceptable acids and, when the compounds can be in the form of optical isomers, the racemic mixture and the individual enantiomers.

A particularly preferred active drug for use in processes and compositions of the invention is tolterodine which has the chemical name (R)-N,N-diisopropyl-3-(2-hydroxy-5-methylphenyl)-3-phenylpropanamine; “tolterodine-related compounds” herein include the corresponding (S)-enantiomer of tolterodine (i.e. (S)-N,N-diisopropyl-3-(2-hydroxy-5-methylphenyl)-3-phenylpropanamine), the racemate, the active 5-hydroxymethyl metabolites (e.g. (R)-N,N-diisopropyl-3-(2-hydroxy-5-hydroxymethylphenyl)-3-phenylpropanamine), and prodrug forms and pharmaceutically acceptable salts thereof.

Such compounds have anti-cholinergic activity and may be used for treating, inter alia, urinary disorders including overactive bladder. The overactive bladder condition gives rise to urinary frequency, urgency and/or urge incontinence. Overactive bladder disorders also include nocturia, i.e. awakening at night to urinate. While overactive bladder is often associated with detrusor muscle instability, disorders of bladder function may also be due to neuropathy of the central nervous system (detrusor hyperreflexia) including spinal cord and brain lesions, such as multiple sclerosis and stroke. Overactive bladder symptoms may also result from, for example, male bladder outlet obstruction (usually due to prostatic hypertrophy), interstitial cystitis, local edema and irritation due to focal bladder cancer, radiation cystitis due to radiotherapy to the pelvis, and cystitis. Such compounds also have anti-spasmodic activity and may be useful in treating gastrointestinal disorders, including gastrointestinal hyperactivity.

Polymer-Based Release-Controlling Component

Compositions of the invention comprise a polymer-based release-controlling component. A “polymer-based release-controlling component” herein is any composition which comprises at least one polymer and which, when applied to and/or or incorporated into a pharmaceutical dosage unit can slow, extend or delay release of therapeutic agent from the dosage unit. “Release of therapeutic agent from a dosage unit” can be determined using a standard in vitro dissolution assay.

Polymers which are suitable for use in polymer-based release-controlling components according to the invention are generally those polymers which are insoluble in aqueous media and are which are thermoplastic. Preferred polymers include cellulose ethers such as cellulose acetate, cellulose propionate, cellulose butyrate, cellulose acetate butyrate, ethylcellulose, hydroxypropylmethylcellulose, etc. Ethylcellulose is a particularly preferred polymer for use in a polymer-based release-controlling component according to the invention.

Polymer-based release-controlling components are preferably prepared in the form of an aqueous polymer dispersion and, when used in a process of the invention, are preferably applied to a substrate, for example a bead or pellet, by spraying or coating the aqueous polymer dispersion onto the substrate. Illustratively, where the composition being prepared is in the form of a bead, such an aqueous polymer dispersion is sprayed onto the bead during one or more processing steps, for example using a fluid bed processor, and is simultaneously or subsequently dried thereon. The term “aqueous polymer dispersion” herein refers to a polymer-based release-controlling component that is in the form of an aqueous dispersion. Such an aqueous polymer dispersion comprises a plurality of polymer particles dispersed in a continuous aqueous phase. The dispersion preferably contains at least one pharmaceutically acceptable plasticizing agent (also referred to as a plasticizer).

Illustrative plasticizers include carboxylic acids (e.g. fatty acids) and salts thereof, alkyl esters of carboxylic acids, in particular C₁-C₆ alkyl esters of fatty acids or C₁-C₄ alkyl esters of phthalic or sebacic acid, propylene glycol, castor oil, medium chain triglycerides (MCT, e.g. coconut oil), etc.

Preferred plasticizers include dibutylsebacate, propylene glycol, triethylcitrate, tributylcitrate, castor oil, acetylated monoglycerides, acetyl triethylcitrate, acetyl butylcitrate, diethyl phthalate, dibutyl phthalate, triacetin, MCT, palmitic acid, oleic acid, stearic acid, linoleic acid, linolenic acid, ricinoleic acid, arachidonic acid, and palmitoleic acid. Oleic acid, MCT and dibutylsebacate are particularly preferred plasticizers.

One suitable aqueous polymer dispersion is Aquacoat® of FMC Corp. Aquacoat® is prepared by dissolving ethylcellulose in a water-immiscible organic solvent and then emulsifying the same in water in the presence of a surfactant and a stabilizer. After homogenization to generate submicron droplets, the organic solvent is evaporated under vacuum to form a pseudolatex. The plasticizer is not incorporated in the pseudolatex during the manufacturing phase. Thus, prior to using the same as a coating, it is desirable to intimately mix the Aquacoat® with a suitable plasticizer.

Another suitable aqueous polymer dispersion is commercially available as Surelease® (Colorcon, Inc.). Surelease® is prepared by incorporating plasticizer into the dispersion during the manufacturing process as is disclosed in U.S. Pat. No. 4,502,888, hereby incorporated by reference herein in its entirety. A hot melt of a polymer, plasticizer (e.g. MCT), and stabilizer (e.g. oleic acid) is prepared as a homogeneous mixture, which is then diluted with an alkaline solution (ammoniated water) to obtain an aqueous polymer dispersion which can be applied directly onto substrates. The term Surelease® herein refers to products, of any grade, marketed under the trade-name, illustratively Surelease® E-7-19010, Surelease® E-7-7050, and Surelease® E-7-19000. Surelease® and equivalents thereto are preferred aqueous polymer dispersions for use in processes and compositions of the invention. Surelease® E-7-19010 is a particularly preferred aqueous polymer dispersion.

In one embodiment, the invention provides a commercial-scale process for manufacture of controlled-release dosage units. The process comprises co-formulating tolterodine or a tolterodine-related compound as an active drug and a pharmaceutically acceptable polymer-based release-controlling component. At least about 70%, preferably at least about 80%, more preferably at least about 90%, and still more preferably substantially all or all of the polymer-based release-controlling component used in the process has an age, at time of dosage unit manufacture, which varies by not more than about 180 days, preferably not more than about 120 days, and more preferably not more than about 90 days. The age control element of this embodiment is suitable for any process which includes steps of co-formulating tolterodine or a tolterodine-related compound and a pharmaceutically acceptable polymer-based release-controlling component. Compositions prepared by such a process are a further embodiment of the invention.

A “commercial-scale” process herein is one which results in an amount of finished dosage units, by weight, of about 50 kg or more, preferably about 1000 kg or more, and more preferably about 5000 kg or more. Unless otherwise qualified, a commercial scale process of the invention can be a continuous process, a single-batch process, or a multi-batch process. Preferably, a commercial scale process is one which results in production of at least a substantial portion of, and preferably substantially all dosage units desired for a commercial manufacturing campaign.

The “age at time of dosage unit manufacture” or “age at time of manufacture” of a polymer-based release-controlling component herein is determined as the number of days between preparation of the release-controlling component itself and use of that component in a process for manufacture of a dosage unit of the invention. Preferably, for use in a process of the invention, the polymer-based release-controlling component is in the form of an aqueous polymer dispersion. In such a case, the age at time of dosage unit manufacture of a polymer-based release-controlling component is determined as the number of days between preparation of the aqueous polymer dispersion and use of that dispersion in a process of manufacture of the invention. The terms “age” and “calendar age” are used synonymously herein except where the term “age” is otherwise qualified.

Where two or more samples of polymer-based release-controlling component which have different calendar ages are used in a single process of the invention and/or or to prepare a single batch of a composition of the invention, the “age” of the dispersion used in that process or to prepare that batch is calculated as the mass weighted average of the individual polymer dispersion sample ages used. For example, if 100 kg of aqueous polymer dispersion having an age at time of batch preparation of 50 days and 50 kg of aqueous polymer dispersion having an age at time of batch preparation of 75 days are admixed and used to prepare a single batch of a composition of the invention, the age of aqueous polymer dispersion used to prepare that batch would be 58.3 days (((100 kg×50 d)+(50 kg×75 d))+(150 kg)).

A technician preparing a controlled release composition according to a process of the present invention can use any suitable means to ensure that polymer-based release-controlling component used in an inventive process has an age, at time of dosage unit manufacture, which varies by not more than a desired number of days. Illustratively, such a technician can standardize age of the aqueous polymer dispersion at time of dosage unit manufacture, for example by monitoring age of aqueous polymer dispersion used to prepare each dosage unit. Such a technician will illustratively record the date of preparation of all lots of aqueous polymer dispersion which will subsequently be used in a process of manufacture of the invention. Before using an aqueous polymer dispersion in such a process, the technician will ensure that the aqueous polymer dispersion has the desired calendar age; the effect of such a monitoring procedure is to ensure that age of aqueous polymer dispersion varies throughout the manufacturing process by not more than a pre-selected number of days, for example not more than about 180 days.

In another embodiment, the invention provides an improved multi-batch process for preparing a controlled release pharmaceutical composition comprising co-formulating tolterodine or a tolterodine-related compound and a polymer-based release-controlling component. The improvement comprises a step of standardizing age, at time of batch preparation, of the polymer-based release-controlling component such that age varies among at least about 70%, preferably at least about 80%, more preferably at least about 90%, and still more preferably substantially all of the batches by not more than about 180 days, preferably not more than about 120 days, and more preferably not more than about 90 days. Preferably such a process is a commercial scale process.

A “batch” in the present context is an amount of finished dosage units of uniform specified quality produced according to a single manufacturing order during a same cycle of manufacture. A cycle of manufacture can result in a batch of any scale, typically about 25 kg to about 3000 kg of finished dosage units. A “multi-batch process” herein is a process for preparing dosage units that is performed in more than one cycle of manufacture, each cycle of manufacture resulting in one batch of dosage units.

Age standardizing according to the present embodiment is suitable for any process which includes steps of co-formulating tolterodine or a tolterodine-related compound and a pharmaceutically acceptable polymer-based release-controlling component. Compositions prepared by such a process are a further embodiment of the invention. A particularly preferred controlled release composition prepared according to a multi-batch process of this embodiment is a controlled release bead, for example as is described herein below.

In another embodiment, the invention provides a commercial-scale process for manufacture of controlled release dosage units. The process comprises co-formulating tolterodine or a tolterodine-related compound and a pharmaceutically acceptable polymer-based release-controlling component. The process further comprises a step of standardizing effective age of the polymer-based release-controlling component such that at least about 70%, preferably at least about 80%, more preferably at least about 90%, and still more preferably substantially all of the polymer-based release-controlling component used in said process is of substantially the same effective age. Particularly preferably, at least about 70%, more preferably at least about 80%, even more preferably at least about 90%, and still more preferably substantially all or all of the polymer-based release-controlling component used in such a process also has a calendar age of about 40 to about 250 days. Compositions prepared according to such a process are a further embodiment of the invention.

It will be understood that two or more samples of polymer-based release-controlling component having the same calendar age can have different effective ages. The “effective age” of a polymer-based release-controlling component or aqueous polymer dispersion is determined by, inter alia, calendar age as described herein above, and by temperature and humidity conditions under which the polymer-based release-controlling component or aqueous polymer dispersion is stored prior to use in a process of the invention. In general, if a first sample of aqueous polymer dispersion is stored at elevated temperature and/or humidity levels by comparison with a second sample of the same aqueous polymer dispersion which has the same calendar age as the first sample, the first sample will be deemed to have a greater effective age.

Illustratively, if two samples of the same aqueous polymer dispersion, Sample A and Sample B, have the same calendar age of 150 days, but Sample A was stored at 40° C. and 75% relative humidity throughout its 150 day life while Sample B was stored under ambient temperature and relative humidity conditions throughout its 150 day life, Sample A will be deemed to have a greater effective age than Sample B. One of ordinary skill in the art will, through routine experimentation, be able to determine whether two or more samples of polymer-based release-controlling component are of substantially the same effective age, for example by use of a test such as that described below.

Whether two or more samples of aqueous polymer dispersion are of substantially the same effective age can illustratively be determined according to Test I.

Test I:

A. A first sample of aqueous polymer dispersion and a second sample of aqueous polymer dispersion are provided;

B. In a first batch, a first controlled release composition comprising an active agent and aqueous polymer dispersion from the first sample is prepared;

C. The first composition is placed in a standard in vitro dissolution test;

D. Three hours after placement in the test, amount of active agent released from the first composition is determined using HPLC;

E. In a second batch, a second controlled release composition is prepared which comprises the same active agent as in the first composition, but which comprises aqueous polymer dispersion from the second sample, (other than presence of aqueous polymer dispersion from different samples, the second composition is identical to the first composition);

F. The second composition is placed in an in vitro dissolution test identical to that utilized in Step C;

G. Three hours after placement in the test, amount of active agent released from the second composition is determined using HPLC;

H. If the amount of active agent released from the first composition at 3 hours is within about ±20% of the amount of active agent released from the second composition, the aqueous polymer dispersion from the first sample and from the second sample are deemed to be of substantially the same effective age; if the amount of drug released from the first composition is more than about ±20% different from the amount of drug released from the second composition, the aqueous polymer dispersion from the first sample and from the second sample are deemed to be of substantially different effective age.

In yet another embodiment, the invention provides a multi-batch process for preparing a controlled release pharmaceutical composition comprising co-formulating tolterodine or a tolterodine-related compound and a polymer-based release controlling component. The process further comprises a step of standardizing effective age, at time of batch preparation, of the polymer-based release-controlling component such that the effective age among at least about 70%, preferably at least about 80%, more preferably at least about 90%, and more preferably substantially all or all of said batches is substantially the same. Effective age standardization according to this embodiment is suitable for any process for preparing a controlled release pharmaceutical composition which comprises co-formulating tolterodine or a tolterodine-related compound and a polymer-based release controlling component. Compositions prepared according to such a process are a further embodiment of the invention.

In a particularly preferred embodiment, the invention provides a controlled release pharmaceutical composition in the form of discrete dosage units having been prepared in a multi-batch process of manufacture; preferably in a commercial scale process of manufacture. The dosage units comprise tolterodine or a tolterodine-related compound as an active drug and a pharmaceutically acceptable polymer-based release-controlling component. The polymer-based release-controlling component has an age distribution at time of manufacture of the dosage units such that upon randomly sampling a plurality of the dosage units and individually placing each of the sampled dosage units in identical standard in vitro dissolution tests, the sampled dosage units exhibit drug release, measured immediately after 3 hours in said dissolution tests, which varies by not more than about ±15%, preferably not more than about ±12.5%, and more preferably not more than about ±10% of a target.

The term “randomly sampling” a plurality of dosage units in the present context refers to drawing of a random sample of dosage units from a pool of like, discrete dosage units, for example having been prepared in a plurality of batches. Any suitable random sampling method can be used. One of skill in the art will readily select an appropriate sampling method based on the number of batches of dosage units being sampled, the form, shape and size of the dosage units, and other factors.

A “target” can represent any desired amount of drug released from a dosage unit and will typically be expressed as a percentage of total drug initially present in a dosage unit (i.e. prior to dissolution testing). For example, if 30 mg of drug is initially present (labeled) in each dosage unit and it is desired that 21 mg of drug is dissolved (released) after 3 hours in a standard dissolution test, then the target will be pre-set at 70% (21 mg/30 mg). A target is preferably established prior to dissolution testing. If a target is not pre-established, the target will, by default, be deemed the median of the dissolution points of all dosage forms tested. If an even number of dosage units are tested, the median will be the mean of the two middle values. Illustrative targets are 50%, 55% or 60% of drug released at 3 hours in a standard dissolution test.

The term “age distribution” herein refers to both calendar age and effective age as defined hereinabove. The term “calendar age distribution” herein refers to the calendar age of a polymer-based release-controlling component and assumes that all polymer-based release-controlling component under consideration was stored under substantially the same temperature and humidity conditions.

An illustrative standard in vitro dissolution test comprises USP apparatus 1 operating at 100 rpm with 900 ml de-aerated 0.05M phosphate buffer at pH 6.8 and 37° C. A dosage unit is placed in the buffer and drug concentration is measured over a period of about 7 hours by HPLC with UV detection at 254 nm.

Preferably, upon individual placement of a random sample of dosage units prepared in a same commercial scale or multi-batch process of the invention in a standard in vitro dissolution test, the dosage units exhibit drug release, measured at 3 hours, such that at least about 80%, preferably at least about 90%, and more preferably substantially all or all of said sampled dosage units release an amount of drug which varies by not more than about ±15%, preferably not more than about ±12.5, and more preferably not more than about ±10, of a target.

The polymer-based release-controlling component used in preparation of at least about 70%, preferably at least about 80%, more preferably at least about 90%, and still more preferably substantially all or all of the dosage units prepared in a same multi-batch or commercial scale process of the invention preferably has an age, at time of dosage unit preparation, which varies by about 180 days or less, preferably by about 120 days or less, more preferably by about 90 days or less.

In another preferred embodiment, the polymer-based release-controlling component used in preparation of at least about 70%, preferably at least about 80%, more preferably at least about 90%, and still more preferably substantially all or all of the dosage units prepared in a same multi-batch or commercial scale process of the invention has a calendar age at time of batch preparation of about 10 to about 190 days, preferably about 40 to about 175 days, and more preferably about 40 to about 125 days.

A exemplary controlled release composition which can be prepared using any of the age standardization or age control steps described herein is a controlled release bead. An illustrative process for preparing such a bead comprises the steps of: (a) providing a core unit of substantially water-soluble or water-swellable material; (b) applying a first layer of a substantially water-insoluble polymer to said core; (c) applying onto said first layer a second layer comprising tolterodine or a tolterodine-related compound and optionally a polymer binder; and (d) applying onto said second layer a third polymer layer comprising the aqueous polymer dispersion; wherein the amount of material in said first layer is selected to provide a layer thickness that permits control of water penetration into the core.

Compositions prepared according to such a process represent a further embodiment of the invention. In such a composition, the core unit comprises any pharmaceutically acceptable excipient which can be molded to form a bead or pellet. Preferably, the core comprises sucrose and/or starch (e.g. sugar spheres NF), sucrose crystals, microcrystalline cellulose, lactose, etc. Preferably, the core unit is in the shape of a sphere and has a diameter of about 0.5 to about 2 mm.

The substantially water-insoluble polymer present in the first layer is preferably insoluble in gastrointestinal fluids. Non-limiting examples of suitable polymers for use in the first layer include ethylcellulose, cellulose acetate, cellulose acetate butyrate, polymethacrylates such as ethyl acrylate/methyl methacrylate copolymer (e.g. Eudragit® NE-30-D) and ammonio methacrylate copolymer types A and B (e.g. Eudragit® RL-30-D and RS-30-D), and silicone elastomers. Preferably, a plasticizer is also present in the first layer. Illustratively, the first layer can include a component comprising both a polymer and one or more plasticizers (e.g. Surelease®). The first layer preferably constitutes about 2% to about 80%, and more preferably about 3% to about 80%, of the total bead weight.

The second layer comprises tolterodine or a tolterodine-related compound as the active ingredient and optionally a polymer binder. The polymer binder, when present, is preferably hydrophilic but may be water-soluble or water-insoluble. Illustrative polymer binders for use in the second layer are hydrophilic polymers such as polyvinylpyrrolidone (PVP), polyalkylene glycols such as polyethylene glycol, gelatin, polyvinyl alcohol, starch and derivatives thereof, cellulose derivatives such as hydroxypropylmethylcellulose (HPMC), hydroxypropylcellulose, hydroxyethylcellulose, carboxyethylcellulose, carboxymethylhydroxyethylcellulose, acrylic acid polymers, polymethacrylates, etc. Preferably the second layer constitutes about 0.05% to about 60%, and more preferably about 0.1% to about 30%, of the total bead weight.

The third layer comprises a polymer-based release-controlling component as described hereinabove. Preferably, the third layer constitutes about 1% to about 50%, and more preferably about 2% to about 25%, of the total bead weight. Optionally, a bead according to this embodiment can further comprise a fourth layer to prevent agglomeration and sticking of individual beads (i.e. a coating layer). Such a coating layer can comprise a polymer or any other desired coating material. A preferred coating material is HPMC. A particularly preferred dosage unit according to this embodiment comprises a plurality of beads encapsulated in a hard capsule, for example a hard gelatin capsule.

### EXAMPLES

The following examples illustrate aspects of the invention but should not be construed as limitations.

### Example 1

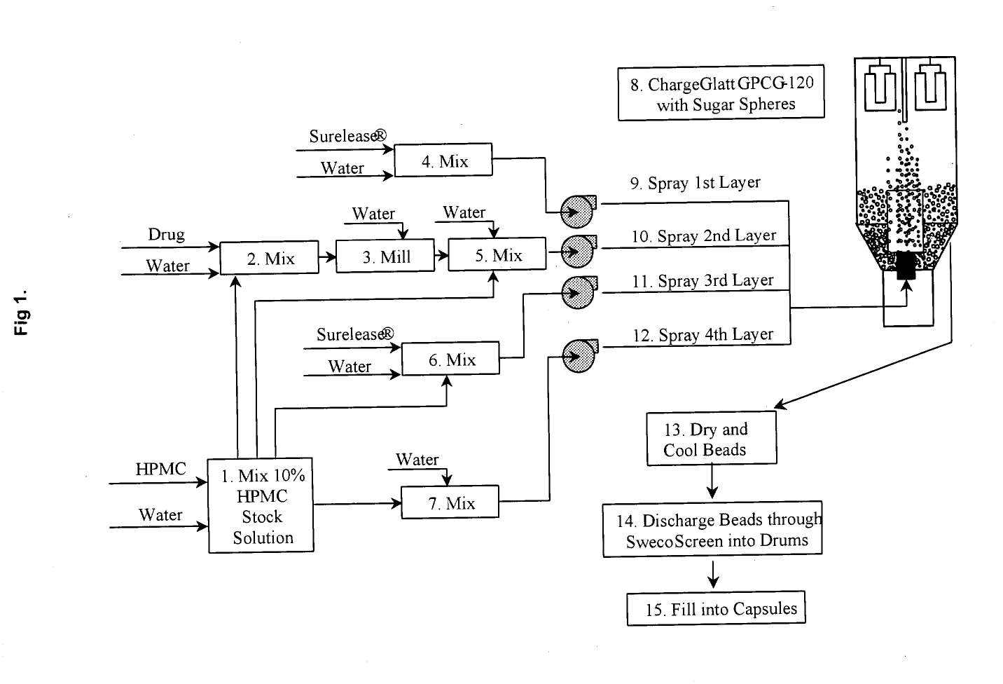

Fifty-seven batches (Batches 1-57) of tolterodine controlled release beads were prepared according to the procedure described immediately below (shown in detail by flow diagram in FIG. 1). In general, various functional layers were prepared as aqueous dispersions and applied to sugar spheres in a Glatt model GPCG-120 fluid bed processor fitted with a 32-inch Wurster column. Tolterodine was wet-milled to reduce its particle size and to distribute it uniformly throughout the resulting mixture. Spray nozzles located at the base of the Wurster column applied dispersions to spheres as they moved through the column, entrained in a high velocity air stream. The spheres exited the top of the column, where they dried as they returned under the influence of gravity to the base of the column to become eventually re-entrained in the high velocity air stream. The re-circulating motion of the spheres continued until the desired amount of dispersion had been applied. Prior to discharge, the coated spheres were dried for approximately 1 hour with approximately 2450 cubic feet per minute airflow at 70° C. The dried beads were then passed through a screen to remove unwanted aggregates.

Age of aqueous polymer dispersion used to prepare each batch was recorded; all batches were prepared with aqueous polymer dispersion which had a calendar age, at time of batch preparation, of 66 to 277 days.

Beads prepared in Batches 1-57 had the composition shown in Table 1. Ninety mg of beads from each batch were then loaded into separate 2 mg hard gelatin capsules (or 180 mg of beads into 4 mg capsules) to form test articles. Test articles individually comprising beads from each of the batches were then placed in an in vitro dissolution test under the following conditions: USP apparatus 1 operating at 100 rpm with 900 ml de-aerated 0.05M phosphate buffer at pH 6.8 and 37° C.; drug concentration was measured by HPLC with UV detection at 254 nm. In some cases, beads were not loaded into capsules prior to in vitro dissolution testing; instead, 200 mg of beads were placed into the dissolution test in unencapsulated form (referred to herein as “in process” dissolution testing).

A target release, by percent weight of drug released after 3 hours in the dissolution test, was pre-set. In total, samples from 42 of the 57 (74%) batches released an amount of drug at 3 hours which was within ±15% of the pre-set target (standard deviation of 12.8%).

TABLE 1
Composition (%) of a coated bead prepared in Batches 1-57

| Component | Dry Weight |
|---|---|
| Core | |
| Sugar spheres | 73.3 |
| First layer | |
| Surelease ® E-7-19010 Clear | 11.9 |
| Purified Water | qs⁽¹⁾ |
| Second layer | |
| Tolterodine L-Tartrate | 2.2 |
| HPMC 2910 | 1.7 |
| Purified Water | qs⁽¹⁾ |
| Third layer | |
| Surelease ® E-7-19010 Clear | 8.5 |
| HPMC 2910 | 1.4 |
| Purified Water | qs⁽¹⁾ |
| Fourth layer | |
| HPMC 2910 | 1.0 |
| Purified Water | qs⁽¹⁾ |

⁽¹⁾Water removed during processing to a residual level of 6% loss on drying or less typically 1%).

### Example 2

Twenty batches (Batches 58-77) of tolterodine controlled release beads were prepared according to the general procedure described in Example 1. All twenty batches were prepared with aqueous polymer dispersion having a calendar age, at time of batch preparation, of 167 to 179 days.

Beads prepared in Batches 58-77 had the composition shown in Table 2. Ninety mg of beads from each batch were then loaded into separate 2 mg hard gelatin capsules (or 180 mg of beads into 4 mg capsules) to form test articles. Test articles individually comprising beads from each of the twenty batches were then placed in the in vitro dissolution test described in Example 1.

A target release, by percent weight of drug released after 3 hours in the dissolution test, was pre-set. In total, samples from 20 of the 20 (100%) batches released an amount of drug at 3 hours which was within ±15% of the pre-set target (standard deviation of 5.5%).

TABLE 2
Composition (%) of a coated bead prepared in Batches 58-63

| Component | Dry Weight |
|---|---|
| Core | |
| Sugar spheres | 73.3 |
| First layer | |
| Surelease ® E-7-19010 Clear | 11.9 |
| Purified Water | qs⁽¹⁾ |
| Second layer | |
| Tolterodine L-Tartrate | 2.2 |
| HPMC 2910 | 1.7 |
| Purified Water | qs⁽¹⁾ |
| Third layer | |
| Surelease ® E-7-19010 Clear | 8.5 |
| HPMC 2910 | 1.4 |
| Purified Water | qs⁽¹⁾ |
| Fourth layer | |
| HPMC 2910 | 1.0 |
| Purified Water | qs⁽¹⁾ |

⁽¹⁾Water removed during processing to a residual level of 6% loss on drying or less typically 1%).

### Example 3

One hundred twenty-nine batches (Batches 78-206) of tolterodine controlled release beads were prepared according to the general procedure described in Example 1. Age of aqueous polymer dispersion used to prepare each batch was recorded; all batches were prepared with aqueous polymer dispersion which had a calendar age, at time of batch preparation, of 56 to 138 days.

Beads prepared in Batches 78-206 had the composition shown in Table 3. Ninety mg of beads from each batch were then loaded into separate 2 mg hard gelatin capsules (or 180 mg of beads into 4 mg capsules) to form test articles. Test articles individually comprising beads from each of the 129 batches were then placed in the in vitro dissolution test described in Example 1.

A target release, by percent weight of drug released after 3 hours in the dissolution test, was pre-set. In total, samples from 126 of the 129 (˜98%) batches released an amount of drug at 3 hours which was within ±15% of the pre-set target (standard deviation of 6.9%).

TABLE 3
Composition (%) of coated beads prepared in batches 64-101

| Component | Dry Weight |
|---|---|
| Core | |
| Sugar spheres | 74.0 |
| First layer | |
| Surelease ® E-7-19010 Clear | 11.4 |
| Purified Water | qs⁽¹⁾ |
| Second layer | |
| Tolterodine L-Tartrate | 2.2 |
| HPMC 2910 | 1.8 |
| Purified Water | qs⁽¹⁾ |
| Third layer | |
| Surelease ® E-7-19010 Clear | 8.1 |
| HPMC 2910 | 1.4 |
| Purified Water | qs⁽¹⁾ |
| Fourth layer | |
| HPMC 2910 | 1.0 |
| Purified Water | qs⁽¹⁾ |

⁽¹⁾Water removed during processing to a residual level of 6% loss on drying or less (typically 1%).

### Example 4

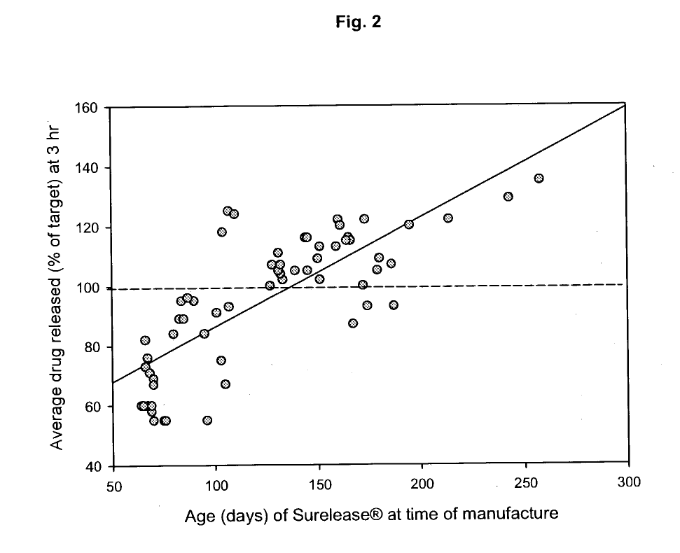

In vitro dissolution data collected from test articles comprising beads prepared in Batches 1-63 of Examples 1 and 2 are plotted as a function of age of aqueous polymer dispersion used to prepare each batch. As shown in FIG. 2, there is a positive correlation between age of aqueous polymer dispersion, at time of batch preparation, and in vitro drug release by 3 hours.

What is claimed is:

1. A controlled release pharmaceutical composition in the form of discrete dosage units having been prepared in a multi-batch process of manufacture, the dosage units comprising: (a) tolterodine or a tolterodine-related compound as an active drug and (b) a pharmaceutically acceptable polymer-based release-controlling component having an age distribution at time of said manufacture such that upon randomly sampling a plurality of the dosage units and individually placing each of said sampled dosage units in identical standard in vitro dissolution tests, the sampled dosage units exhibit drug release, measured immediately after 3 hours in said dissolution tests, that varies by not more than about ±15% of a target.

2. The composition of claim 1 wherein the pharmaceutically acceptable polymer-based release-controlling component has an age distribution at time of said manufacture such that upon randomly sampling a plurality of the dosage units and individually placing each of said sampled dosage units in identical standard in vitro dissolution tests, the sampled dosage units exhibit drug release, measured immediately after 3 hours in said dissolution tests, that varies by not more than about ±12.5% of a target.

3. The composition of claim 1 wherein the pharmaceutically acceptable polymer-based release-controlling component has an age distribution at time of said manufacture such that upon randomly sampling a plurality of the dosage units and individually placing each of said sampled dosage units in identical standard in vitro dissolution tests, the sampled dosage units exhibit drug release, measured immediately after 3 hours in said dissolution tests, that varies by not more than about ±10% of a target.

4. The composition of claim 1 wherein the polymer-based release-controlling component has a calendar age at time of said manufacture of about 10 to about 190 days.

5. The composition of claim 1 wherein the polymer-based release-controlling component has a calendar age at time of said manufacture of about 40 to about 175 days.

6. The composition of claim 1 wherein the polymer-based release-controlling component has a calendar age at time of said manufacture of about 40 to about 125 days.

7. The composition of claim 1 wherein the polymer-based release-controlling component has a calendar age at time of said manufacture that varies by not more than about 180 days.

8. The composition of claim 1 wherein the polymer-based release-controlling component has a calendar age at time of said manufacture that varies by not more than about 90 days.

9. The composition of claim 1 wherein the polymer-based release-controlling component used to prepare substantially all of the batches is of substantially the same effective age at time of said manufacture.

10. The composition of claim 1 wherein the polymer-based release-controlling component comprises ethylcellulose and at least one plasticizer.

11. The composition of claim 10 wherein the at least one plasticizer comprises fractionated coconut oil.

12. The composition of claim 1 wherein the polymer-based release-controlling component comprises Surelease® or an equivalent thereto.

13. The composition of claim 1 wherein the polymer-based release-controlling component comprises Aquacoat® or an equivalent thereto.

14. The composition of claim 1 wherein the drug is tolterodine.

15. A commercial scale, multi-batch process for preparing a controlled release pharmaceutical composition comprising co-formulating (a) tolterodine or a tolterodine-related compound and (b) a polymer-based release-controlling component; wherein age, at time of batch preparation, of the polymer-based release-controlling component is standardized such that said age varies among substantially all of the batches by not more than about 180 days.

16. The process of claim 15 wherein age, at time of batch preparation, of the polymer-based release-controlling component is standardized such that said age varies among substantially all of the batches by not more than about 120 days.

17. The process of claim 15 wherein age, at time of batch preparation, of the polymer-based release-controlling component is standardized such that said age varies among substantially all of the batches by not more than about 90 days.

18. The process of claim 15 wherein age, at time of batch preparation, of the polymer-based release-controlling component is standardized such that said age varies among all the batches by not more than about 90 days.

19. The process of claim 15 wherein effective age, at time of batch preparation, of the polymer-based release-controlling component is standardized such that said effective age is substantially the same among all the batches.

20. The process of claim 15 wherein the polymer-based release-controlling component comprises ethylcellulose and at least one plasticizer.

21. The process of claim 20 wherein the plasticizer comprises fractionated coconut oil.

22. The process of claim 15 wherein the polymer-based release-controlling component comprises Surelease® or an equivalent thereto.

23. The process of claim 15 wherein the polymer-based release-controlling component comprises Aquacoat® or an equivalent thereto.

24. The process of claim 15 wherein the drug is tolterodine.

25. A pharmaceutical composition prepared according to the process of claim 15.

26. A commercial scale, multi-batch process for preparing a controlled release bead comprising the steps of:
(a) providing a core unit of substantially water-soluble or water-swellable material;
(b) applying a first layer of a substantially water-insoluble polymer to said core;
(c) applying onto said first layer a second layer comprising tolterodine or a tolterodine-related compound and optionally a polymer binder; and
(d) applying onto said second layer a third polymer layer comprising an aqueous polymer dispersion;
wherein the amount of material in said first layer is selected to provide a layer thickness that permits control of water penetration into the core; and
(e) standardizing age, at time of batch preparation, of the aqueous polymer dispersion such that the age varies among substantially all of said batches by not more than about 180 days.

27. The process of claim 26 wherein age, at time of batch preparation, of the aqueous polymer dispersion is standardized such that said age varies among substantially all of the batches by not more than about 120 days.

28. The process of claim 26 wherein age, at time of batch preparation, of the aqueous polymer dispersion is standardized such that said age varies among substantially all of the batches by not more than about 90 days.

29. The process of claim 26 wherein the aqueous polymer dispersion comprises ethylcellulose and at least one plasticizer.

30. The process of claim 29 wherein the plasticizer comprises fractionated coconut oil.

31. A pharmaceutical composition comprising 1 to a plurality of controlled release beads prepared according to the process of claim 26 enclosed in a capsule.

32. A method of treating a medical condition or disorder in subject where treatment with an anti-cholinergic agent is indicated, comprising orally administering to the subject a composition of claim 1, once or twice per day.

33. A method of treating a medical condition or disorder in subject where treatment with an anti-cholinergic agent is indicated, comprising orally administering to the subject a composition of claim 25, once or twice per day.

34. A method of treating a medical condition or disorder in subject where treatment with an anti-cholinergic agent is indicated, comprising orally administering to the subject a composition of claim 31, once or twice per day.
